(19) United States
(12) Patent Application Publication
(10) Pub. No.: US 2022/0309590 A1
(43) Pub. Date: Sep. 29, 2022

(54) SYSTEMS AND METHODS FOR DOCUMENT MANAGEMENT

(71) Applicant: Change Healthcare Holdings, LLC, Nashville, TN (US)

(72) Inventors: Suprigya Babu, Redmond, VA (US); Harshit Patel, Cumming, GA (US); Peter Corazao, Sammamish, WA (US); Sang Huynh, Auburn, WA (US); Greg Jones, Suwanee, GA (US); Alka Mukker, Duluth, GA (US)

(21) Appl. No.: 17/215,168

(22) Filed: Mar. 29, 2021

(51) Int. Cl. G06Q 40/08; G06F 16/25; G16H 10/60; G16H 40/20; G06F 40/103

(52) U.S. Cl. CPC G06Q 40/08; G06F 16/258; G16H 10/60; G16H 40/20; G06F 40/103

(57) ABSTRACT

In one embodiment, a document management system is provided that automatically converts documents submitted electronically with or for a claim into a format that is required by a third-party payor. The document management system maintains a list of document format and delivery preferences for each third-party payor. Rather than submit a claim and document directly to the third-party payor, the submitter submits the claim and document to the document management system using a portal that accepts a wide variety of electronic document formats. The document management system then retrieves the format and delivery preferences of the third-party payor and converts the documents into whatever format is required by the third-party payor. The document management system then delivers the converted document to the third-party payor according to the delivery preferences such as email, fax, or regular mail.

### BACKGROUND

In an example environment, processing payment for medical claims, documents (also referred to herein as “attachments”) supporting the claim are often required by payers. Some, but not all, payers will accept electronic documents. Others will require actual hard copy documents. Thus, submitters/providers cannot or will not all submit attachments required for processing the payment electronically. Approximately 5% of claims may require an attachment. Even at the 5% rate, some 170 million claim attachments are sent manually (mail or fax) each year. That is, approximately 80% of attachments are sent using manual processes like mail and fax. This is costly.

Handling attachments manually is costly. Providers currently spend an average of $4.50 to send attachments manually. Providers spend an average of 11 minutes per attachment when handled manually vs. 5 minutes when sent via an electronic method. Uncertainty around attachments standards have kept software vendors and payers on the sideline as the manual attachment problem has persisted. Accordingly, there is a need for an attachments solution that is easy to integrate, provides access to nearly any payer and that can evolve with emerging and changing standards.

### SUMMARY

In one embodiment, a document management system is provided that automatically converts documents submitted electronically with or for a claim into a format that is required by a third-party payor. The document management system maintains a list of document format and delivery preferences for each third-party payor. Rather than submit a claim and document directly to the third-party payor, the submitter submits the claim and document to the document management system using a portal that accepts a wide variety of electronic document formats. The document management system then retrieves the format and delivery preferences of the third-party payor and converts the documents into whatever format is required by the third-party payor. The document management system then delivers the converted document to the third-party payor according to the delivery preferences such as email, fax, or regular mail.

In an embodiment, a method for managing document formats and delivery is provided. The method includes: receiving a document in a first data format of a plurality of formats by a computing device, wherein the document is associated with a matter identifier and a third-party payor; determining document preferences associated with the third-party payor by the computing device, wherein the document preferences identify a second data format of the plurality of formats and one or more document delivery preferences; converting the document to the second data format by the computing device; and providing the document to the third-party in the second data format by the computing device according to the document delivery preferences.

Embodiments may include some or all of the following features. Receiving the document may include receiving the document through one or more of an application program interface or a portal application. The document may be received in a batch along with a plurality of documents. The document may be received in a 277 document format. The matter identifier may be a claim identifier, and the document may be received in support of an insurance claim for the third-party payor. The method may further include: converting the document from the first data format to a third data format, wherein the third data format is an intermediate data format; and storing the document in the third data format in a datastore, and wherein converting the document to the second data format may further include: retrieving the document from the datastore; and converting the document from the third data format to the second data format. The delivery preferences may include one or more of a preference for mail delivery, a preference for fax delivery, a preference for electronic delivery, a preference for a batch delivery, and a preference for delivery through a portal associated with the third-party payor. The document may be received from a submitter, and the method may further include: receiving a communication associated with the document from the third-party payor, wherein the communication is associated with the matter identifier; based on the matter identifier, determining the submitter that provided the document; and providing the communication to the submitter. The communication may indicate that the document was accepted or rejected by the third-party payor. The third-party payor may be a medical insurance provider.

The document management system described herein provides many advantages over the prior art. First, by converting documents automatically into document formats that are required by third-party payors, claims submitters are free to use whatever document format they prefer including native document formats. This saves each submitter time as they do not have to convert documents or remember the different formats required by each third-party payor. The system also saves the third-party payors time by reducing the number of rejected or wrongly-formatted documents, and by providing a single entity to deal with when changes are made to a desired format by a third-party payor.

Second, by automatically providing the documents to the third-party payors using desired delivery methods, claims submitters save enormous amounts of time by avoiding having to print, fax, or mail documents. Many submitters almost exclusively deal with electronic documents and electronic document formats making printing and mailing a document a costly and time consuming interruption to their workflows.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate a document attachment system and method. Together with the description, the figures further serve to explain the principles of the document attachment system and method described herein and thereby enable a person skilled in the pertinent art to make and use the document attachment system and method.

FIG. 1 is an example environment for automatically converting and delivering documents according to third-party payor document format and delivery preferences;

FIG. 2 is an illustration of a method for converting and providing documents in response to third-party payor preferences;

FIG. 3 is an illustration of a method for providing communications received from a third-party payor to a submitter; and

FIG. 4 shows an exemplary computing environment in which example embodiments and aspects may be implemented.

### DETAILED DESCRIPTION

Reference will now be made in detail to embodiments of the document attachment system and method with reference to the accompanying figures. The same reference numbers in different drawings may identify the same or similar elements.

The present systems and methods provide an attachment/document solution that can be integrated into present systems and provides access to nearly any payer/third-party. While described herein with respect to medical submission environment, attachment submission described herein and the described architectures, systems and methods are applicable in any environment in which documentary evidence is submitted.

Reference will now be made in detail to embodiments of the document attachment system and method with reference to the accompanying figures. The same reference numbers in different drawings may identify the same or similar elements.

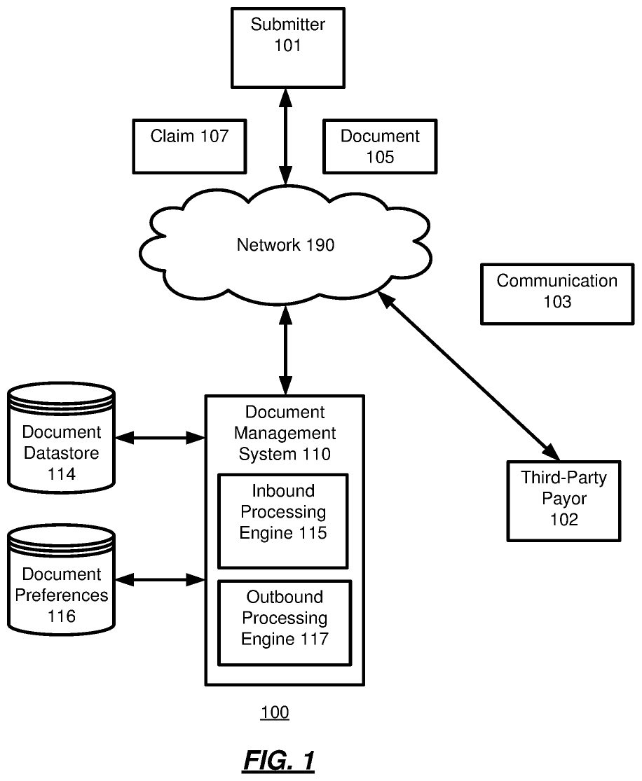

FIG. 1 is an example environment 100 for automatically converting and delivering documents according to third-party payor document format and delivery preferences. As shown, the environment 100 includes a submitter 101, a third-party payor 102, and a document management system 110 communicating through a network 190. While only one submitter 101 and third-party payor 102 are shown, it is for illustrative purposes only; it is contemplated that there may be multiple submitters and third-party payors 102 in environment 100.

The submitter 101 may include medical providers, individuals, or any other entity that may submit a claim 107 or request to a third-party payor 102. The third-party payor 102 may be a third-party payor in the business of receiving claims 107 from submitters 101 and either paying the claims 107, denying the claims 107, or requesting additional information about the claim 107. Examples of third-party payors 102 include insurance companies (e.g., medical insurance payors), and government agencies (e.g., unemployment providers, or Medicaid payors).

Generally, when a claim 107 is submitted by submitter 101 to a third-party payor 102, it may include one or more supporting documents 105. These documents 105 are often provided as an attachment to a corresponding claim 107 and are sometimes referred to as attachments. The documents 105 may include a variety of documents and document types that may support the corresponding claim 107 such as medical reports (e.g., doctor reports or orders), medical images (e.g., X-rays, MRIs, and cat scans), and other types of evidence that may support a claim 107 (e.g., affidavits, photographs, or videos).

As described above, many third-party payors 102 have various rules or requirements about the formats and delivery methods that they require for supporting documents 105. For example, some third-party payors 102 require that documents 105 be physically mailed or faxed to a particular address or phone number and be accompanied by a particular form or coversheet that links it to the associated claim 107. Other third-party payors 102 may allow for electronic submission of documents but may have specific requirements on the format or structure of the document 105. For example, some third-party payors 102 may want certain document 105 fields in a certain order or in particular units (e.g., inches vs cm), or may require that document 105 images be provided in certain formats (jpeg vs. bitmaps), resolutions (500×500 vs. 300×300), or sizes (e.g., no documents 105 greater than 5 mb).

Even where electronic documents 105 are accepted, some payors 102 may require that the documents 105 be uploaded through a specific portal, or be provided to a specific email address, for example. As may be appreciated, complying with the document 105 requirements of third-party payors 102 is a difficult and cumbersome endeavor.

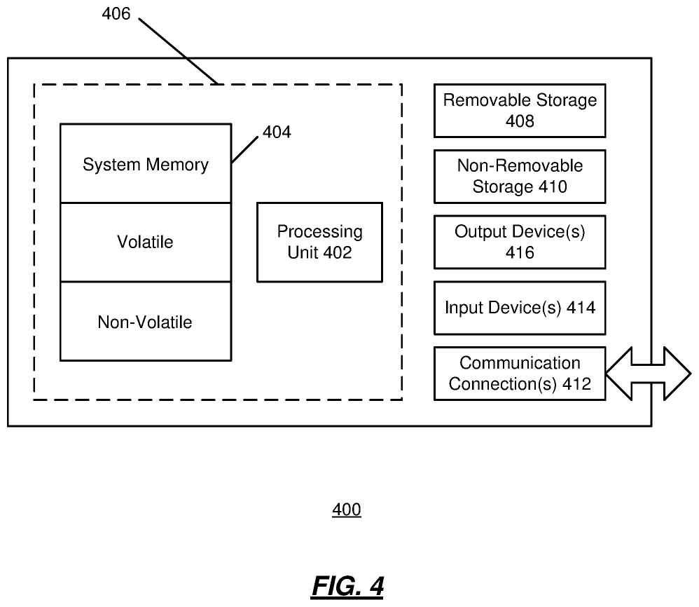

Accordingly, to automatically convert and deliver documents 105 according to third-party payor 102 preferences, the environment 100 may include a document management system 110. As shown, the document management system 110 may include various components including an inbound processing engine 115; and an outbound processing engine 117. More or fewer components may be supported. Depending on the embodiment, each of the engines 115 and 117 may be implemented together or separately using one or more general purpose computing devices such as the computing device 400 illustrated with respect to FIG. 4. In addition, either engine may be implemented together or separately using a cloud-based computing environment.

The inbound processing engine 115 may receive claims 107 and documents 105 from one or more submitters 101 through a network 190. The documents 105 that support a claim 107 may be received at the same time as the corresponding claim 107 (e.g., as an attachment) or may be received at a later time or date. Each document 105 may have a matter identifier (also referred to as claim identifier) that links it to a particular claim 107. Note that depending on the implementation, while the documents 105 may be received by the inbound processing engine 115, claims 107 may be received and handled by an entity or component other than the inbound processing engine 115.

In some embodiments, the inbound processing engine 115 may provide an electronic portal through which the submitters 101 may provide claims 107 and one or more more documents 105 in an electronic format. In other embodiments, the inbound processing engine 115 may expose an application programming interface (“API”) that the submitters 101 may use to electronically provide claims 107 and documents 105. For example, the submitters 101 may use various functions provided or exposed by the API to submit documents 105 and/or claims 107 to the inbound processing engine 115. The API may allow submitters 101 to use their existing programs and/or applications to interface with the inbound processing engine 115 and/or the document management system 110.

The submitters 101 may be able to use the portal or API to view the status of their claims 107 and documents 105. For example, the API may expose information about each document 105 such as when it was delivered to a third-party payor 102, whether it was accepted by the third-party payor 102, and whether the third-party payor 102 has requested any follow-up information or has provided a communication 103 in response to a document 105. Such communications 103 are described further below.

In some embodiments, the submitter 101 may submit claims 107 and/or documents 105 to the inbound processing engine 115 using fax or by physically mailing the claim 107 and/or document 105 to a phone number or address associated with the inbound processing engine 115. The inbound processing engine 115 (or another service or entity) may receive the claims 107 and/or documents 105 and may convert them into a suitable electronic format (e.g., may scan or manually enter information from the claims and/or documents 105).

The inbound processing engine 115 may receive claims 107 and/or documents 105 from submitters 101 sporadically as they are generated. Alternatively, the claims 107 and documents 105 may be received from submitters 101 in batches of multiple transactions. The batches may be provided on a scheduled basis (e.g., a batch every day), or after some amount of claims 107 and/or documents 105 have been generated by the submitter 101 (e.g., every 50 or 100 claims 107 or documents 105).

The inbound processing engine 115 may receive a document 105, either individually or as part of a batch of documents 105 and may convert the document 105 into an intermediate format or structure that is supported by the inbound processing engine 115. The intermediate format may be a proprietary format used by the document management system 110 or may be a common or known format such as but not limited to, JPG, BMP, GIF, TIF, TIFF, PDF, DOC, DOCX, TXT, RTF, JPEG, and PNG. The document 105 may be received in a variety of formats including EDI 277. Other formats may be supported.

As part of the format conversion, the inbound processing engine 115 may convert the document 105 into a standard size or resolution and may optionally compress or encrypt the document 105 for storage in a document datastore 114. The inbound processing engine 115 may store the document 105 in the document datastore 114 along with the matter identifier of the associated claim 107, and an identifier of the third-party payor 102 that the document 105 is directed to. In some embodiments, each document 105 may be stored as a JavaScript Object Notation (“JSON”) object. Other formats may be supported.

Note that the conversion of the document 105 into the intermediate format before storage in the document datastore 114 is strictly optional. In some embodiments, the documents 105 may be stored in the document datastore 114 in the same format that they were received in from the submitter 101. Converting and storing the documents 105 in the intermediate format may result in improved storage efficiency and improved performance when ultimately converting the documents 105 into the format preferred by the third-party payor 102 for delivery.

In some embodiments, the inbound processing engine 115 may validate the documents 105 that are received from the submitter 101. Document validation may include determining the accuracy or authenticity of the received documents 105.

In some embodiments, the inbound processing engine 115 may further match or associate the received documents 105 with any previously received claims 107. As may be appreciated, documents 105 may be submitted later than the corresponding claim 107, or in response to a request or communication 103 from a third-party payor 102. Accordingly, when the document 105 is received the inbound processing engine 115 may match the document 105 to a corresponding claim 107 based on the matter numbers associated with the document 105 and the claim 107.

The outbound processing engine 117 may take a document 105 from the document datastore 114 and may provide the document 105 to the associated third-party payor 102 in a format that is specified by the third-party payor 102 and using a delivery means specified by the third-party payor 102.

The outbound processing engine 117 may store or maintain document preferences 116 for each of the third-party payors 102. The documents preferences 116 for a third-party payor 102 may specify the format and contents of the documents 105 that are expected by the third-party payor 102. The format of the document 105 may specify the particular file type or file format that is expected by the payor 102 and may include a variety of formats such as BMP, GIF, TIF, TIFF, PDF, DOC, DOCX, TXT, RTF, JPEG, and PNG. Other formats may be supported. The format of the document may also specify requirements such as a minimum or maximum resolution, a maximum file size, and whether or not the document 105 should be encrypted (as well as the public key that may be used to encrypt the document 105) or compressed.

The document preferences 116 may further specify the various data fields that should be included in the document 105 and may include the order in which they are expected in the document 105. These fields may include patient name, date, matter or claim identifier, and other descriptive information about the document 105. Other information may be included.

The document preferences 116 for a third-party payor 102 may further indicate the delivery preferences of the third-party payor 102. The preferences may be for electronic delivery and may indicate an email address, FTP address, or electronic portal that should be used for delivery of the document 105. The preferences 116 may further indicate that the documents 105 should be physically mailed and may include an address that should be used. The preferences 116 may indicate that the document 105 should be faxed and may include a fax number that should be used.

The document preferences 116 may further include information such as whether or not the third-payor 102 wants documents 105 to be provided as they are received or batched into larger groups of documents 105. The preferences 116 may indicate the number of documents 105 in each batch and/or the frequency at which batches of document 105 may be provided.

In some embodiments, the document preferences 116 may be provided to the outbound processing engine 117 by the third-party payors 102. For example, the outbound processing engine 117 may expose an API or may provide a portal through which the third-party payors 102 may upload their document preferences 116 or may edit or change existing document preferences 116. Alternatively, the document preferences 116 for a third-party payor 102 may be provided by the submitter 101 or may be extracted from one or more documents or contracts provided by the third-party payor 102 and/or submitter 101.

In some embodiments, the document preferences 116 for a third-party payor 102 may vary depending on the type of document 105 or even the type of claim 107 that is associated with the document 105. For example, for documents 105 that are medical images such as X-rays, the document preferences 116 may specify that the document 105 be in the TIFF format, and for documents 105 that are affidavits or police reports the document preferences 116 may specify that the document 105 be in the PDF format.

When a document 105 is selected for processing by the outbound processing engine 117 from the document datastore 114, the outbound processing engine 117 may retrieve the document preferences 116 for the third-party payor 102. The outbound processing engine 117 may then convert or transform the document 105 into the format that is specified by the document preferences 116. Depending on the embodiment, the document 105 may be converted from the format the document 105 was received in from the submitter 101, or the intermediate format that was used to store the document 105 in the document datastore 114.

After the document 105 is converted, the outbound processing engine 117 may attend to transmitting or sending the converted document 105 to the associated third-party payor 102 according to the document preferences 116. Where the preferences 116 are to electronically send the document 105, the outbound processing engine 117 may use the network 190 to electronically deliver the document 105 using whatever methods are preferred by the payor 102 (e.g., e-mail, ftp, or electronic portal). If directed by the preferences 116, the outbound processing engine 117 may include a coversheet or whatever additional materials are desired by the third-party payor 102.

Where the document preferences 116 indicate that the document 105 should be faxed, the outbound processing engine 117 may facilitate the faxing of the document 105 (and preparation of the coversheet) to the payor 102 at the indicated fax number. Depending on the embodiment, the engine 117 may use an electronic document faxing service or may arrange for a user or employee to physically fax the converted document 105.

Where the document preferences 116 indicate that the document 105 should be sent by physical mail, the outbound processing engine 117 may electronically send the document 105 (and an associated letter or coversheet) to an employee or other individual that is contracted to mail documents 105 on behalf of the document management system 110. The employee or individual may then print the document 105 and related materials and may mail the document 105 and related materials to the payor 102 at the specified address. Note that where the documents 105 are sent in batches, the employee or individual may be periodically provided with a batch of documents 105 to mail based on the preferences 116 of the payor 102.

After a document 105 is sent to a third-party payor 102, the outbound processing engine 117 may update metadata associated with the document 105 in the datastore 114 to indicate that the document 105 was sent. Other information such as the date and time that it was sent and the method for sending (e.g., email, fax or mail) may also be included. The submitter 101 may then view the sent status of the document 105 using the API or portal provided by the document management system 110. Alternatively or additionally, the inbound processing engine 115 may send an email or notification to the submitter 101.

In some embodiments, the inbound processing engine 115 may facilitate the transmission of communications 103 from the the third-party payors 102 to the submitters 101 regarding documents 105 or claims 107. With respect to claims 107, the third-party payor 102 may determine that a document 105 is needed for a claim 107 submitted by the submitter 101 and may send a communication 103 requesting the document 105 to the inbound processing engine 115. In response, the inbound processing engine 115 may send the communication 103 to the submitter 101 and may store the communication 103 in the document datastore 114, or another location. Later, when a document 105 is received from the submitter 101, the inbound processing engine 115 may determine that the document 105 is in response to the communication 103 and may mark the communication 103 as having been resolved.

With respect to documents 105, the third-party payor 102 may determine that there are one or more issues associated with a document 105 and may send a communication 103 requesting a new or revised document 105 to the inbound processing engine 115. For example, the document 105 may have been of a low quality, may not have supported the associated claim 107, or may have been in the wrong format. In response, the inbound processing engine 115 may send the communication 103 to the submitter 101 and may store the communication 103 in the document datastore 114, or another location. Later, when a corrected document 105 is received from the submitter 101, the inbound processing engine 115 may determine that the corrected document 105 is in response to the communication 103 and may mark the communication 103 as having been resolved.

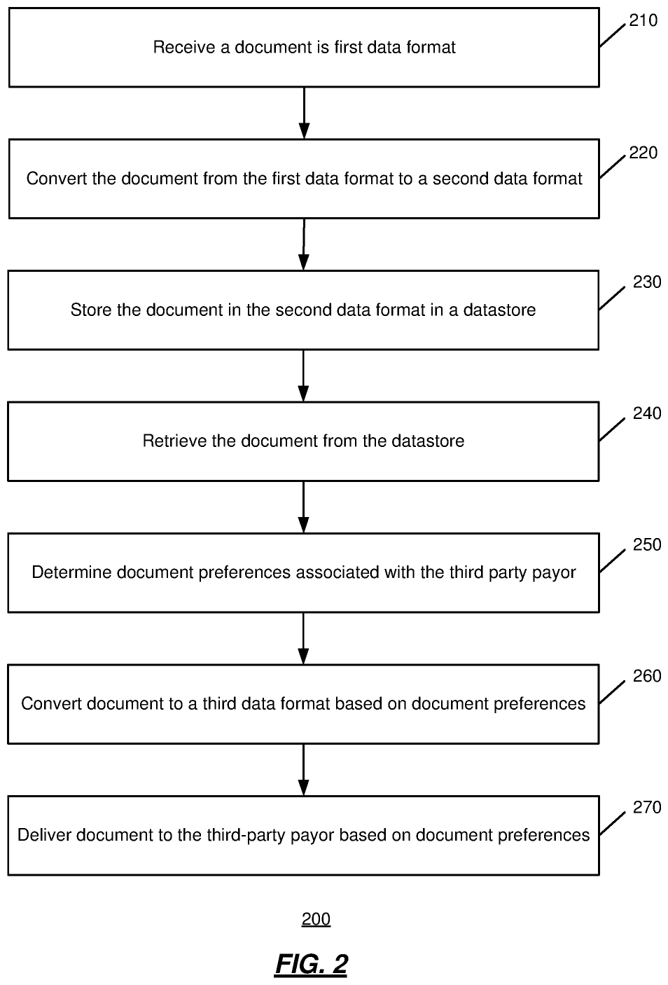

FIG. 2 is an illustration of a method 200 for converting and providing documents in response to third-party payor preferences. The method 200 may be implemented by the document management system 110.

At 210, a document is received in a first data format. The document 105 may be received from a submitter 101 by a document management system 110 through a network 190. The document 105 may be associated with claim 107 and may include a matter identifier that identifies the associated claim 107. The document 105 may support the claim 107 and may include medical images, tests, affidavits, or any other type of document 105 that may be used to support a medical claim 107 or another type of insurance claim. The document 105 may be intended to be ultimately received by a third-party payor 102 who the submitter 101 desires to pay the claim 107. The first data format may be a format that is used by the submitter 101, but that may not be used or supported by the third-party payor 102, such as 277 EDI.

At 220, the document is converted into a second data format. The document 105 may be converted to a second data format by the inbound processing engine 115. The second data format may be an intermediate format that is used by the inbound processing engine 115. The second data format may be a format that can be easily or quickly converted to another format, or that takes up less storage space than the first data format. Any suitable format may be used.

At 230, the the document is stored in a datastore in the second data format. The document 105 may be stored by the inbound processing engine 115 in a datastore such as the document datastore 114. The documents 105 may be stored with the matter identifier of the associated claim 107 and the third-party payor 102 that will receive the document 105. In some embodiments, the inbound processing engine 115 may determine whether the document 105 is received in response to any communications 103 received from the third-party payor 102, and if so, may mark the communication 103 as having been satisfied or responded to.

At 240, the document is retrieved from the datastore. The document 105 may be retrieved from the document datastore 114 by the outbound processing engine 117 soon after the document 105 was received, or as part of a batch processing of documents 105 that are to be delivered to the associated third-party payor 102.

At 250, document preferences associated with the third-party payor are determined. The document preferences 116 may be determined by the outbound processing engine 117. In some embodiments, the document preferences 116 for a third-party payor 102 may have been provided by the third-party payor 102.

At 260, the document is converted to a third data format based on the document preferences. The document 105 may be converted by the outbound processing engine 117 according to the format specified by the document preferences 116. As part of the conversion, in some embodiments, the outbound processing engine 117 may rearrange certain fields of the document 105, may add or remove one or more fields from the document 105, or may change the size or resolution of the document 105 according to the document preferences 116. Any method for converting a document format may be used.

At 270, the document is delivered to the third-party payor based on the document preferences. The document 105 may be delivered by the outbound processing engine 117 to the third-party payor 102 using the delivery method specified in the document preferences 116. Depending on the embodiment, the outbound processing engine 117 may deliver the document in third data format using an electronic delivery method (e.g., e-mail or FTP), or a non-electronic delivery method (e.g., fax, mail, or courier). After the document 105 is delivered, the outbound processing engine 117 may mark or update the document 105 in the document datastore 114 to indicate that the document 105 was delivered.

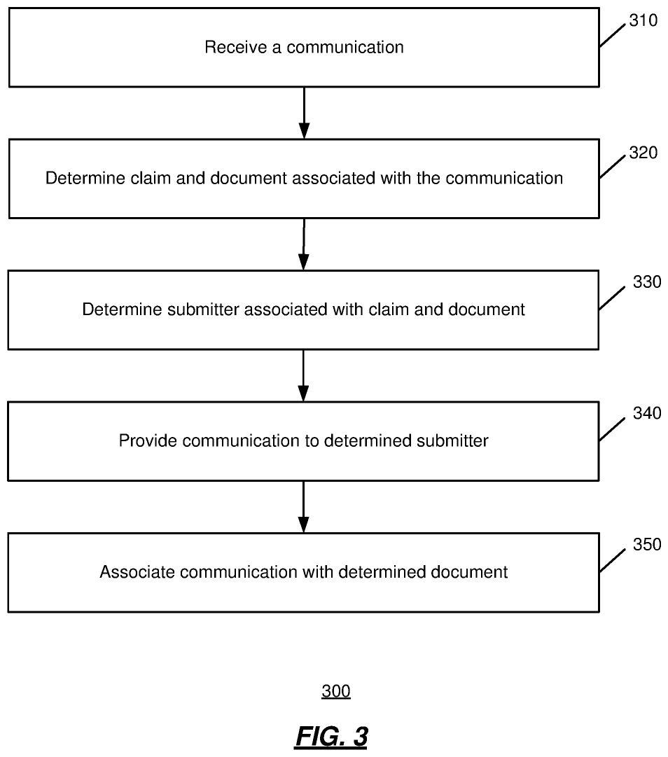

FIG. 3 is an illustration of a method 300 for providing communications received from a third-party payor to a submitter. The method 300 may be implemented by the document management system 110.

At 310, a communication is received. The communication 103 may be received by the inbound processing engine 115 from a third-party payor 102. The communication 103 may relate to a document 105 previously provided to the third-party payor 102 by the inbound processing engine 115. The communication 103 may indicate that the document 105 was accepted by the third-party payor 102 or that there are one or more issues or problems with the document 105 that was provided.

At 320, a claim and document associated with the communication is determined. The claim 107 and document 105 may be determined by the inbound processing engine 115. Depending on the embodiment, the communication 103 may identify the document 105 and/or the claim 107 that the communication pertains to, or may include an identifier (e.g., matter identifier) that the inbound processing engine 115 may use to determine the claim 107 and document 105.

At 330, a submitter associated with the claim and document is determined. The inbound processing engine 115 may identify the submitter 101 using information associated with the determined document 105 and/or claim 107 in the document datastore 114 or other data storage used by the inbound processing engine 115.

At 340, the communication is provided to the determined submitter. The communication 103 may be provided to the submitter 101 by the outbound processing engine 117. The communication 103 may be provided through the network 190 via a portal provided by the document management system 110, an API exposed by the document management system 110 to one or more applications used by the submitter 101 or may be emailed to submitter 101. Any method for communicating electronically may be used.

At 350, the communication is associated with the determined document. The communication may be associated with the document 105 in the document datastore 114. For example, the outbound processing engine 117 may store the communication 103 with the document 105 or may otherwise mark the document 105 in the document datastore 114 to show that there is an outstanding communication 103. Once it is determined that the submitter 101 has responded to the communication (if necessary), the communication 103 and/or mark may be removed from the document datastore 114.

FIG. 4 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 4, an exemplary system for implementing aspects described herein includes a computing device, such as computing device 400. In its most basic configuration, computing device 400 typically includes at least one processing unit 402 and memory 404. Depending on the exact configuration and type of computing device, memory 404 may be volatile (such as random access memory (RAM)), non-volatile (such as read-only memory (ROM), flash memory, etc.), or some combination of the two. This most basic configuration is illustrated in FIG. 4 by dashed line 406.

Computing device 400 may have additional features/functionality. For example, computing device 400 may include additional storage (removable and/or non-removable) including, but not limited to, magnetic or optical disks or tape. Such additional storage is illustrated in FIG. 4 by removable storage 408 and non-removable storage 410.

Computing device 400 typically includes a variety of computer readable media. Computer readable media can be any available media that can be accessed by the device 400 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 404, removable storage 408, and non-removable storage 410 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 400. Any such computer storage media may be part of computing device 400.

Computing device 400 may contain communication connection(s) 412 that allow the device to communicate with other devices. Computing device 400 may also have input device(s) 414 such as a keyboard, mouse, pen, voice input device, touch input device, etc. Output device(s) 416 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method for managing document formats and delivery comprising:
receiving a document in a first data format of a plurality of formats by a computing device, wherein the document is associated with a matter identifier and a third-party payor;
determining document preferences associated with the third-party payor by the computing device, wherein the document preferences identify a second data format of the plurality of formats and one or more document delivery preferences;
converting the document to the second data format by the computing device; and
providing the document to the third-party in the second data format by the computing device according to the document delivery preferences.

2. The method of claim 1, wherein receiving the document comprises receiving the document through one or more of an application program interface or a portal application.

3. The method of claim 1, wherein the document is received in a batch along with a plurality of documents.

4. The method of claim 1, wherein the document is received in a 277 document format.

5. The method of claim 1, wherein the matter identifier is a claim identifier, and the document is received in support of an insurance claim for the third-party payor.

6. The method of claim 1, further comprising:
converting the document from the first data format to a third data format, wherein the third data format is an intermediate data format; and
storing the document in the third data format in a datastore, and wherein converting the document to the second data format comprises:
retrieving the document from the datastore; and
converting the document from the third data format to the second data format.

7. The method of claim 1, wherein the delivery preferences comprise one or more of a preference for mail delivery, a preference for fax delivery, a preference for electronic delivery, a preference for a batch delivery, and a preference for delivery through a portal associated with the third-party payor.

8. The method of claim 1, wherein the document is received from a submitter, and further comprising:
receiving a communication associated with the document from the third-party payor, wherein the communication is associated with the matter identifier;
based on the matter identifier, determining the submitter that provided the document; and
providing the communication to the submitter.

9. The method of claim 8, wherein the communication indicates that the document was accepted or rejected by the third-party payor.

10. The method of claim 1, wherein the third-party payor is a medical insurance provider.

11. The method of claim 1, wherein the document preferences are received from the third-party payor.

12. A system for managing documents and document providers, the system comprising:
at least one computing device; and
a memory storing computer-executable instructions that when executed by the at least one computing device cause the at least one computing device to:
receive a document in a first data format of a plurality of formats, wherein the document is associated with a matter identifier and a third-party payor;
determine document preferences associated with the third-party payor, wherein the document preferences identify a second data format of the plurality of formats and one or more document delivery preferences;
convert the document to the second data format; and
provide the document to the third-party in the second data format according to the document delivery preferences.

13. The system of claim 12, wherein receiving the document comprises receiving the document through one or more of an application program interface or a portal application.

14. The system of claim 12, wherein the document is received in a batch along with a plurality of documents.

15. The system of claim 12, wherein the document is received in a 277 document format.

16. The system of claim 12, wherein the matter identifier is a claim identifier, and the document is received as part of an insurance claim for the third-party payor.

17. The system of claim 12, further comprising:
converting the document from the first data format to a third data format, wherein the third data format is an intermediate data format; and
storing the document in the third data format in a datastore, and wherein converting the document to the second data format comprises:
retrieving the document from the datastore; and
converting the document from the third data format to the second data format.

18. The system of claim 17, wherein the delivery preferences comprise one or more of a preference for mail delivery, a preference for fax delivery, a preference for electronic delivery, a preference for a batch delivery, and a preference for delivery through a portal associated with the third-party payor.

19. The system of claim 12, wherein the document is received from a submitter, and further comprising:
receiving a communication associated with the document from the third-party payor, wherein the communication is associated with the matter identifier;
based on the matter identifier, determining the submitter that provided the document; and
providing the communication to the submitter.

20. The system of claim 19, wherein the communication indicates that the document was accepted or rejected by the third-party payor.
